(19) United States
(12) Patent Application Publication
(10) Pub. No.: US 2020/0305878 A1
(43) Pub. Date: Oct. 1, 2020

(54) USER INTERFACE FOR DEPLOYING FILAMENT COILS

(71) Applicant: Spiration, Inc., d/b/a Olympus Respiratory America, Redmond, WA (US)

(72) Inventor: David A. Herrin, Seattle, WA (US)

(73) Assignee: Spiration, Inc., d/b/a Olympus Respiratory America, Redmond, WA (US)

(21) Appl. No.: 16/367,154

(22) Filed: Mar. 27, 2019

(51) Int. Cl. A61B 17/11

(52) U.S. Cl. CPC A61B 17/1114; A61B 1/018

(57) ABSTRACT

Disclosed embodiments include apparatuses and methods for deploying a coilable filament on opposing sides of a surface. An apparatus includes a needle mechanism configured to extend and withdraw the needle. A coil mechanism is configured to advance a stylet through the needle to extend a coilable filament at a distal end of the stylet. An interlock mechanism is configured to control operations of the needle mechanism and the coil mechanism, including moving the needle and the stylet in concert to extend the needle and coilable filament to a distal side of the surface, separately advancing the stylet to extend a first segment of the coil on the distal side of the surface, withdrawing the needle to the proximal side of the surface, and separately further advancing the stylet to deploy a second segment of the coil along the proximal side of the surface.

### FIELD

The present disclosure relates to a user interface for deploying coilable filaments on opposing sides of a surface.

### BACKGROUND

The statements in this section merely provide background information related to the present disclosure and may not constitute prior art.

Blockages of internal ducts or other openings within a living body, such as a blockage of the common bile duct, may result in serious consequences. Bile is released by the liver to aid the digestive system in breaking down fats. Bile is stored in the common bile duct for release into the small intestine. The common bile duct provides a passageway to carry bile from the common bile duct into the upper portion of the small intestine. A blockage of the common bile duct, which may be caused by tumors, cysts, scar tissue, infection, inflammation, or any number of causes, prevents bile from being released into the small intestine. Blockage of the common bile duct not only prevents the bile from reaching the small intestine, but also results in a build-up of bile in the liver. The build-up of bile may result in pain, a build-up of bilirubin in the blood causing jaundice, and other health problems.

When the common bile duct becomes blocked, a new passage may be formed between the common bile duct and the small intestine to allow for bile to flow into the small intestine. However, conventional abdominal surgery is painful, debilitating, and, as with any invasive surgery, potentially risky, especially for patients with compromised health. Minimally invasive techniques to create a new passageway between the common bile duct and the small intestine pose difficulty because of the location of the organs and the delicate nature of the tissues. Particularly problematic is being able to form a sealed connection between the elastic tissues of the common bile duct and the small intestine prior to cutting a hole to form a new drainage duct. If the tissues are not clamped together prior to creating a hole, bile can leak into the body cavity and a lasting anastomosis will not be formed.

### SUMMARY

Disclosed embodiments include apparatuses for deploying filament coils on opposing sides of a surface and methods for deploying a coilable filament on opposing sides of a surface.

In an illustrative embodiment, an apparatus includes a needle mechanism configured to extend a needle through a surface to a distal side of the surface and to withdraw the needle to a proximal side of the surface. A coil mechanism is configured to advance a stylet through the needle to extend a coilable filament releasably coupled at a distal end of the stylet through the needle, whereupon exiting the needle the coilable filament forms a coil. An interlock mechanism is configured to control operations of the needle mechanism and the coil mechanism. The needle mechanism and the coil control mechanism are movable to advance the needle and the stylet in concert to the distal side of the surface. The coil mechanism is movable to advance the stylet to extend a first segment of the coil on the distal side of the surface. The needle mechanism and the coil mechanism are movable to withdraw the needle to the proximal side of the surface. The coil mechanism is rotatable to further advance the stylet to deploy a second segment of the coil along the proximal side of the surface.

In another illustrative embodiment, an apparatus includes a housing defining a central chamber. A sheath mechanism is configured to enable slidable movement of a sheath housing a needle to a surface and to withdraw the needle from the surface. A needle mechanism having a control handle rotatable and slidable along the housing to extend the needle through the surface to a distal side of the surface and to withdraw the needle to a proximal side of the surface. A depth control mechanism is configured to be lockably positioned along the housing to limit travel of the control handle to limit travel of the needle. A coil mechanism having a coil knob disposed at a proximal end of the housing is rotatable to advance a stylet through the needle to extend a coilable filament releasably coupled at a distal end of the stylet through the needle, whereupon exiting the needle the coilable filament forms a coil. An interlock mechanism is operably coupled with the needle mechanism and the coil mechanism. The needle mechanism and the coil control mechanism are movable to advance the needle and the stylet in concert to the distal side of the surface. The coil mechanism is movable to advance the stylet to extend a first segment of the coil on the distal side of the surface. The needle mechanism and the coil mechanism are movable to withdraw the needle to the proximal side of the surface while rotating the coil mechanism to deploy the first segment of the coil along the distal side of the surface. The coil mechanism is rotatable to further advance the stylet to deploy a second segment of the coil along the proximal side of the surface.

In a further illustrative embodiment, a method comprises a needle movably linked with a stylet received within the needle being extended to cause a tip of the needle to pierce a surface and extend the tip of the needle through the surface to a distal side of the surface while extending the stylet with the needle to extend a coilable filament within the needle to the distal side of the surface. The stylet is movably disengaged from the needle and advanced through the needle to extend a first length of a coilable filament coupled at a distal end of the stylet through the tip of the needle, whereupon exiting the tip of the needle the first length of coilable filament forms a first coil on the distal side of the surface. Simultaneously the needle is retracted and the stylet is rotated to cause the tip of the needle to withdraw to a proximal side of the surface and to deploy the first segment of the coil along the distal side of the surface. The stylet is movably disengaged from the needle and advanced to extend a second length of the coilable filament through the tip of the needle, whereupon exiting the tip of the needle the second length of the coilable filament coils to form a second coil on the proximal side of the surface.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, with emphasis instead being placed upon illustrating the principles of the disclosed embodiments. In the drawings:

FIG. 1 is a schematic diagram of a user interface for deploying coils of a filament on opposing sides of a surface

FIGS. 2A-2C are schematic representations of coils of a filament deposited on opposing sides of a surface using the user interface of FIG. 1;

FIG. 3 is a schematic diagram of the user interface of FIG. 1 coupled with a positioning system;

FIG. 4 is a perspective view in partial cutaway of the user interface of FIG. 1;

FIG. 5 is an exploded view of components of the user interface of FIG. 1;

FIG. 6 is a perspective view in partial cutaway of a sheath mechanism for moving a sheath relative to a surface;

FIGS. 7A and 8A are schematic views of the apparatus of FIG. 1 coupled with the positioning system of FIG. 3 in which the sheath lock is manipulated to permit movement of the sheath;

FIGS. 8B and 8B are schematic views of a distal end of the sheath and other components showing positioning of the sheath corresponding to the manipulations of the user interface as shown in FIGS. 7A and 8A;

FIGS. 9-11 are perspective views of a portion of the housing and components of the depth control mechanism;

FIG. 12 is an exploded view of components of the control handle 130 of FIG. 1;

FIG. 13 is a partial cutaway view of the components of the control handle in engagement with the control knob of FIG. 1;

FIG. 14 is a partial cutaway view of the retraction lock engaged with components of the control handle of FIG. 1;

FIG. 15 is a cross-sectional view of components of the control handle of FIG. 1 showing engagement of pin rockers and a cam plate;

FIG. 16 is a partial cutaway view of a cam tube, cam shaft, and stylet carriage of the user interface of FIG. 1;

FIG. 17 is a side view of the stylet carriage and the stylet of FIG. 16;

FIGS. 18 and 19 are cross-sectional views of a travel stop engaging the stylet carriage of FIGS. 15 and 16;

FIGS. 20A, 21A, 22A, 23A, 24A, 25A, 26A, 27A, 28A, 29A, 30A and 31A are perspective views of an illustrative user interface for deploying coils on opposing sides of a surface;

FIGS. 20B, 21B, 22B, 23B, 24B, 25B, 26B, 27B, 28B, 29B, 30B and 31B are schematic diagrams of positioning of distal ends of a sheath, a needle, and a coilable filament at a surface point corresponding to positions of the components of the user interface of FIGS. 20A, 21A, 22A, 23A, 24A, 25A, 26A, 27A, 28A, 29A, 30A and 31A, respectively; and

FIG. 32 is a flow diagram of an illustrative method of deploying coils on opposing sides of a surface.

### DETAILED DESCRIPTION

The following description is merely illustrative in nature and is not intended to limit the present disclosure, application, or uses. It will be noted that the first digit of three-digit reference numbers, the first two digits of four-digit reference numbers correspond to the first digit of one-digit figure numbers and the first two-digits of the figure numbers, respectively, in which the element first appears.

The following description explains, by way of illustration only and not of limitation, various embodiments of user interfaces and methods to deploy a coilable filament on opposing sides of a surface. As will be described in detail below, apparatuses and methods position a needle to pierce a surface, extend the needle through to a distal side of the surface, extend through the needle a first length of coilable filament to form a first coil on the distal side of the surface, withdraw the needle to a proximal side of the surface, and extend through the needle a second length of the coilable filament to form a second coil on the proximal side of the surface. The surface may include one or more tissues, such as a tissue wall within a body. For a specific example, the surface may be adjacent walls of a common bile duct and a small intestine through which a duct is to be formed. Placement of the first and second coils holds the tissues together, while cutting off blood supply to regions of the tissues surrounded by the coils, causing the regions of tissues to eventually break down to form a fistula between the coils. The first and second coils may then fall away, leaving the fistula in place to permit bile to blow from the common bile duct into the small intestine. Apparatuses herein described provide a user interface to facilitate the process of extending and retracting the needle and extending a coilable filament to form the coils, as well as methods of forming a new duct by deploying coilable filaments. Apparatuses and methods also provide for the separate locking of the needle and the coilable filament to ensure that a proper sequence is followed in moving the needle and the coilable filament.

For purposes of brevity, the following description chiefly addresses, via a non-limiting example given by way of illustration only, use of the apparatuses and methods disclosed herein to create a new opening, commonly called an ampulla, between the common bile duct and the small intestine. However, the process of creating a new opening is provided for the sake of illustration only and not of limitation. It will be appreciated that the apparatuses and methods disclosed herein also may be used to apply coilable filaments to form coils on opposing sides of other surfaces, to join those surfaces, to form openings in other surfaces, and/or for other purposes as desired for a particular application.

By way of introduction and as described further below, in various embodiments, a length of coilable filament is attached at a distal end of a stylet that, in turn, extends through a needle. The needle is extended through a surface (or multiple adjacent surfaces) to a distal side of the surface. A length of coilable filament is partially extended at that point to form a first segment of coil. The needle is then retracted to a proximal side of the surface, where the stylet is turned to cause the first segment of coil to twist to be deployed along the distal side of the surface. A remaining portion of the coilable filament is then extended to form a second segment of coil along a proximal side of the surface. The length of coilable filament is attached to a distal end of the stylet by a weld or similar joint that is strong enough to hold the length of coilable filament in place at the distal end of the stylet but to disengage or break when the weld or joint passes beyond the distal end of the needle. When the length of coilable filament extends beyond the distal end of the needle, the coiling of the length of the coilable filament may apply a lateral strain to the joint, disengaging the length of coilable filament from the stylet, leaving the coil in place.

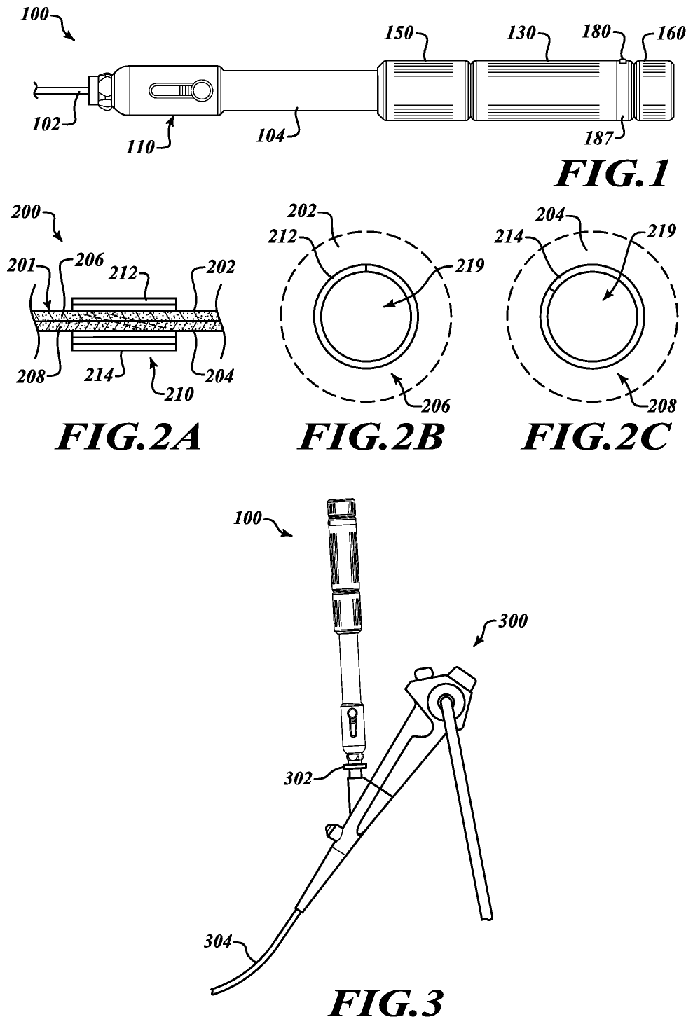

Referring to FIG. 1, a user interface 100 is provided for controlling a needle and a coilable filament extending through the needle (not shown in FIG. 1) contained in a sheath 102. The sheath 102 may be conveyed to a desired location using a positioning system (not shown in FIG. 1) as further described below with reference to FIGS. 3 and 7A-8B. As described in detail below, when the sheath 102 has been conveyed to position the needle to a desired location, the user interface 100 may be used to operate the needle and control extension of the coilable filament extending through the needle. As specifically described herein, for example, the positioning system may convey the sheath 102 into the small intestine where the user interface 100 is used to extend the needle through a wall of the small intestine into a wall of the common bile duct (neither of which is shown in FIG. 1). Once the needle has been extended from the small intestine into the common bile duct, a first length of a coilable filament is extended from the needle. Extension of the coilable filament results in formation of a first coil within the common bile duct. The needle may then be retracted from the common bile duct into the small intestine, where a second length of coilable filament is extended from the needle to form a second coil.

The user interface 100 includes a housing 104 that supports a plurality of mechanisms to extend a sheath 102 containing a needle and a coilable filament (not shown in FIG. 1) to a surface where coils are to be deposited through operation of a number of mechanisms, as further described below. The mechanisms include a sheath mechanism 110, a needle mechanism controlled by operation of a control handle 130, a coil mechanism controlled both by the control handle 130 and a coil knob 160, and an interlock mechanism controlled by the control handle 130 and a retraction lock 180 to prevent unintended withdrawal of the needle. A needle depth control mechanism 150 is movable along the housing 104 to limit travel of the control handle 130 to limit movement of the needle control mechanism to limit a depth to which the needle is inserted. An interlock indicator 187 may provide visual indication of the operational state of the user interface 100, such as whether the user interface 100 is in needle extension mode, coil extension mode, etc. Operation of the sheath mechanism 110, the needle mechanism, the coil mechanism, and the interlock mechanism by user actuation of the control handle 130, the needle depth control mechanism 150, the coil knob 160, the retraction lock 180, and the interlock indicator 187 are described in detail below.

The operation of the mechanisms may be used to deposit coils along a surface, as described with reference to FIGS. 2A-2C. Referring to FIGS. 2A-2C, a region 200 around a surface 201 shows the deployment of coils 210 including a first coil 212 and a second coil 214. In the example of FIGS. 2A-2C, FIG. 2A shows a cross-sectional view of the surface 201, which may include a section of tissue or, in the present example, sections of adjoining tissues 206 and 208. For example, when the surface 201 includes represents adjoining tissues, the tissue may include a portion of a wall of a small intestine 208 that abuts a portion of wall of a common bile duct 206. As is further described below, the coils 212 and 214 are formed by deploying a coilable filament. The coilable filament may include a memory wire, such as nitinol, a nickel-titanium alloy, which may be configured to default to a coiled shape when in an undeformed condition but which assume a linear shape when constrained within a needle used to deploy the coilable filament, as further described below.

Referring to FIGS. 2A and 2B, a first coil 212 is deployed at a distal side 202 of the surface 201, which, for example, includes without limitation the wall of the common bile duct 206. Referring to FIGS. 2A and 2C, a second coil 214 is deployed at a proximal side 204 of the surface 201 which, for example, includes without limitation the wall of the small intestine 208. The second coil 214 represents a continuation of the coilable filament extending from the first coil 212 as it passes through the surface 201. The connected first coil 212 and second coil 214 thus impinge upon the surface from opposing distal and proximal sides 202 and 204, respectively.

Referring to FIGS. 2B and 2C, in an example of living tissue, such as where the surface 201 includes the wall of the common bile duct 206 and the wall of the small intestine 208, deployment of the first coil 212 and the second coil 214 cuts off blood flow to an encircled region 219 of the surface 201 that is surrounded by the first coil 212 and the second coil 214. The coils 212 and 214 hold together walls of the common bile duct 206 and the small intestine 208 and, over time, the loss of blood flow may result in the breakdown of tissue in the encircled region 219. As a result, a new opening is formed at the location of the encircled region 219 in the surface 201. The opening is sealed between the common bile duct 206 and the small intestine 208 to allow a lasting astomosis around the new opening. When the surface 201 includes the wall of the common bile duct 206 and the wall of the small intestine 208, the new opening enables bile to flow from the common bile duct into the small intestine.

In various embodiments, once the opening has formed, it may be desirable for the first coil 212 and the second coil 214 to fall away from the surface 201. Thus, in various embodiments when the surface 201 includes the wall of the common bile duct 206 and the wall of the small intestine 208, it may be desirable that the second coil 214 be larger than the first coil 212. A larger second coil 214 may result by depositing one or more additional revolutions of the coilable filament in forming the second coil 214 as compared with the second coil 212. As a result, once the new opening is formed by the healing together of the contacting tissues and the breakdown of the encircled region 219 of the surface, the connected first coil 212 and second coil 214 may fall into the small intestine where the coils 212 and 214 can be expelled from the body.

Referring to FIG. 3, in order to form the coils 212 and 214 described with reference to FIGS. 2A-2C, it may be desirable to couple the user interface 100 with a positioning system 300 for conveying the sheath 102 to a desired location. The positioning system 300 may include an endoscope or a comparable system that includes a controllable guiding apparatus 304 that can be motivated and manipulated to position a distal end (not shown) of the controllable guiding apparatus 304 to the desired location. In the example of seeking to form an opening to replace a blocked common bile duct, the positioning system may include an endoscope of which the controllable guiding apparatus 304 is inserted into the digestive track and maneuvered into the duodenum or small intestine. The user interface 100 is connected to the positioning system 300 at a port 302 configured to receive the user interface 100. As described below, manipulation of the sheath mechanism 110 to enable movement of the user interface 100 relative to the port 302 enables an operator to position the sheath 102 at the desired location.

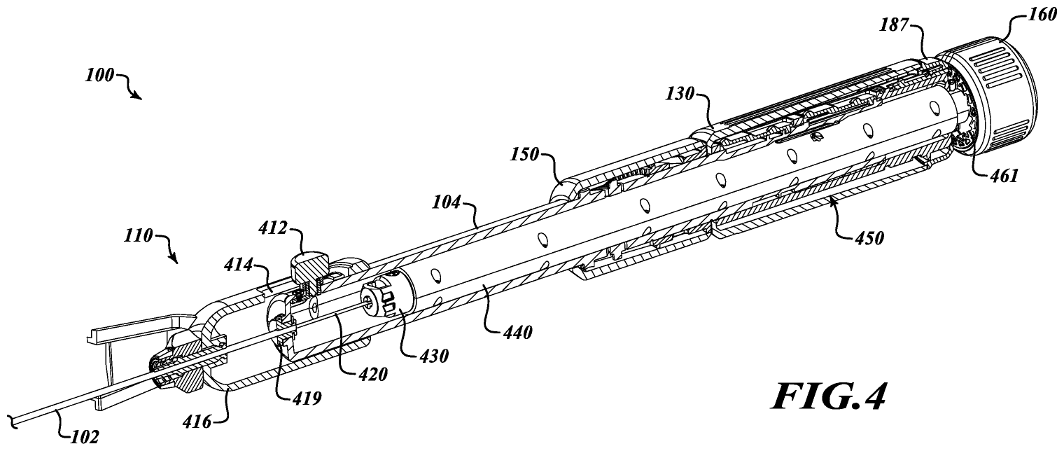

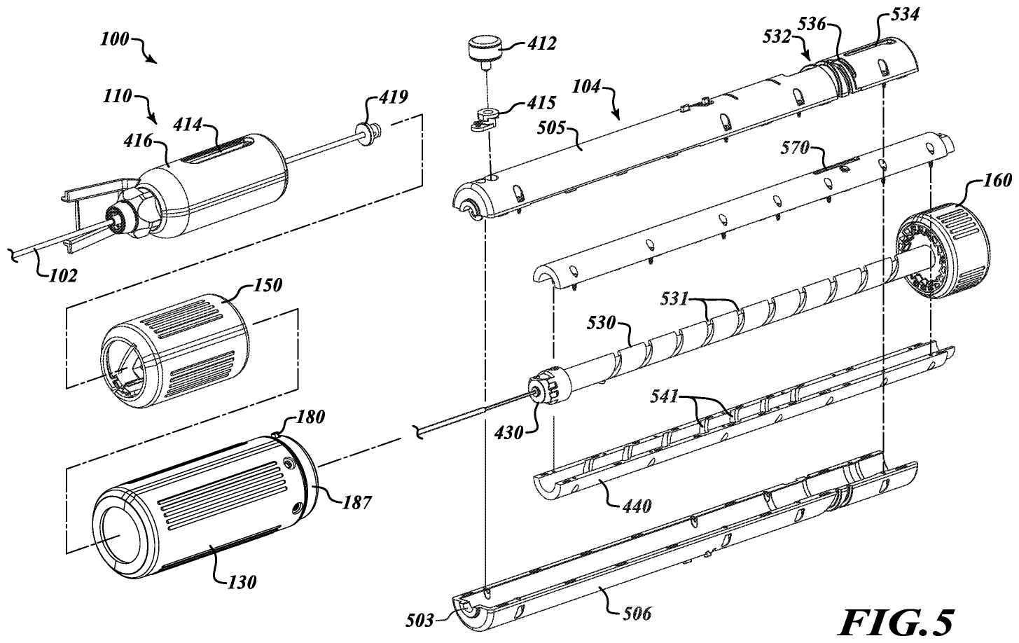

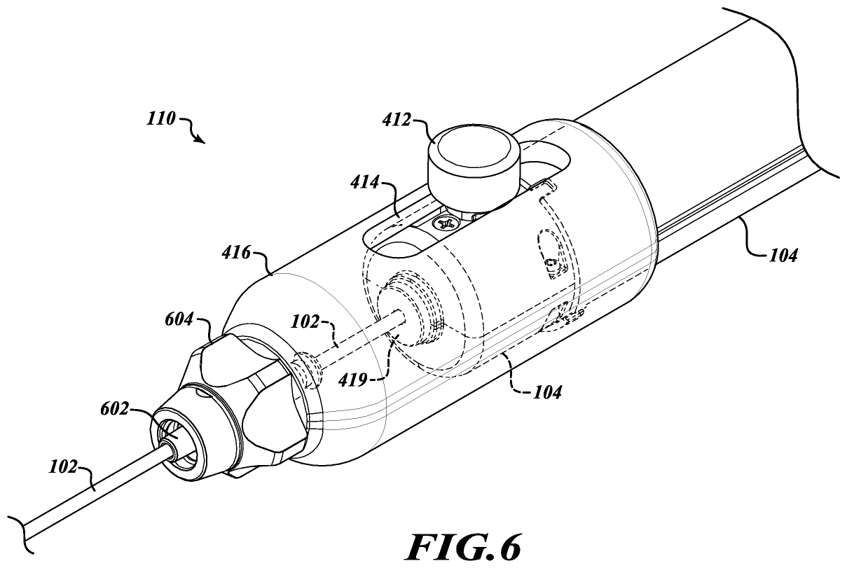

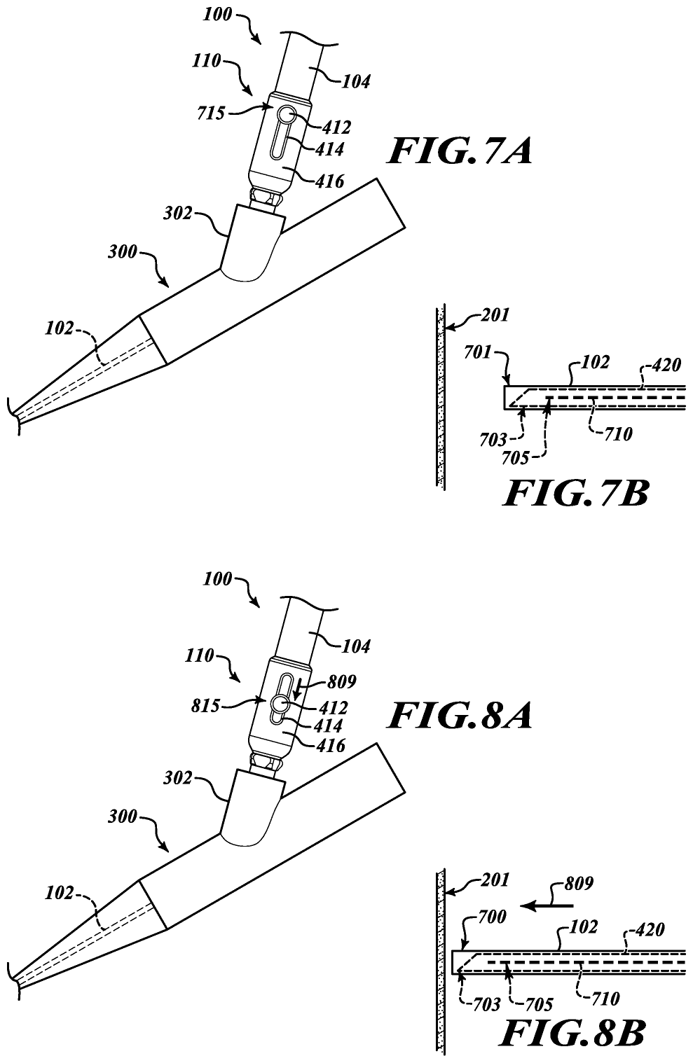

Referring to FIG. 4, components of an embodiment of the user interface 100 are positioned within or about the housing 104. The sheath 102 is coupled the housing 104 at a sheath hub 419. A sheath mechanism 110 is positioned at an end of the housing 104. Operation of the sheath mechanism 110 is controlled by a sheath lock 412 selectively sliding within a channel 414 of a sheath cap 416 enables the housing 104 to slide relative to the sheath cap 416 to advance or retract the sheath 102, as further described below with reference to FIGS. 6-7B.

A needle 420 is slidably received within the sheath 102. The needle 420 is coupled with a cam tube 440 at a thrust bushing 430. The control handle 130 selectively engages the cam tube 440 to extend and withdraw the needle 420. The cam tube 440 houses a cam shaft (not shown in FIG. 4) that is attached to the coil knob 160. Rotation of the coil knob 160 rotates the cam shaft relative to the cam tube 440 to advance a stylet (not shown in FIG. 4) to advance a coilable filament (not shown in FIG. 4) to deploy coils on a surface, as described further below. In various embodiments, the coil knob 160 is coupled to the cam shaft with a ratchet 461 allowing the coil knob 160 to rotate the cam shaft in only a single direction during coil deployment to be able to extend the coilable filament but not to retract the coilable filament, as explained further below. In various embodiments, the coil knob 160 may be slidably detached from the ratchet to allow the coil knob 160 to be turned in an opposite direction to facilitate the retraction of the stylet to load a coilable filament for deployment using the user interface 100.

The control handle 130 engages a handle subassembly 450 that, as part of an interlock mechanism, enables the control handle 130 to slide the control handle 130 to advance the needle 420 and the coilable filament. The handle subassembly 450 also enables the control handle 130 to be disengaged from the cam shaft to enable the coil knob 160 to be rotated to extend the coilable filament without advancing the needle. The handle subassembly 450 also enables the control handle 130 to be reengaged with the cam shaft to enable the control handle 130 to be rotated to withdraw the needle 420 while rotating the shaft to rotate the stylet to twist the coilable filament to position a coil segment along a distal side of a surface, as described further below. An indicator ring 187 marks a position of the control grip 130 to identify a current phase of operation of the user interface 100.

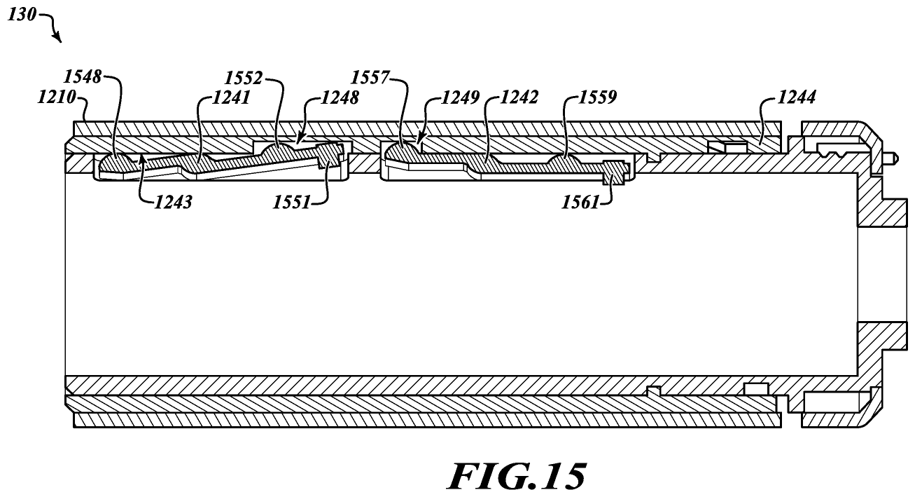

Referring to FIG. 5, components of the user interface in an exploded view include a nut plate 415 couplable to the housing 104 to receive the sheath lock 412 extending through the channel 414 of the sheath cap 416. The housing 440 in various embodiments includes housing sections 505 and 506 that may be molded or otherwise formed to define the structures herein described. The housing sections 505 and 506 define a sheath port 503 into which the sheath hub 419 of the sheath mechanism 110 is received. The housing sections 505 and 506 also support a plurality of control channels 532 that enable the control handle 130 to selectively engage the housing 140 to advance, rotate, and/or retract the housing 104 to move the needle 420 and the stylet (not shown in FIG. 5) to manipulate the needle 420 and the coilable filament. The control channels 532 may include a straight section 534 to guide the control handle 130 in slidable motion relative to the housing 104 and one or more transverse sections 536 to guide the control handle 130 to rotate about the housing 104. The control handle 130 may include one or more inwardly-facing pins, as described further below with reference to FIGS. 12 and 15, configured to engage the control channels 532.

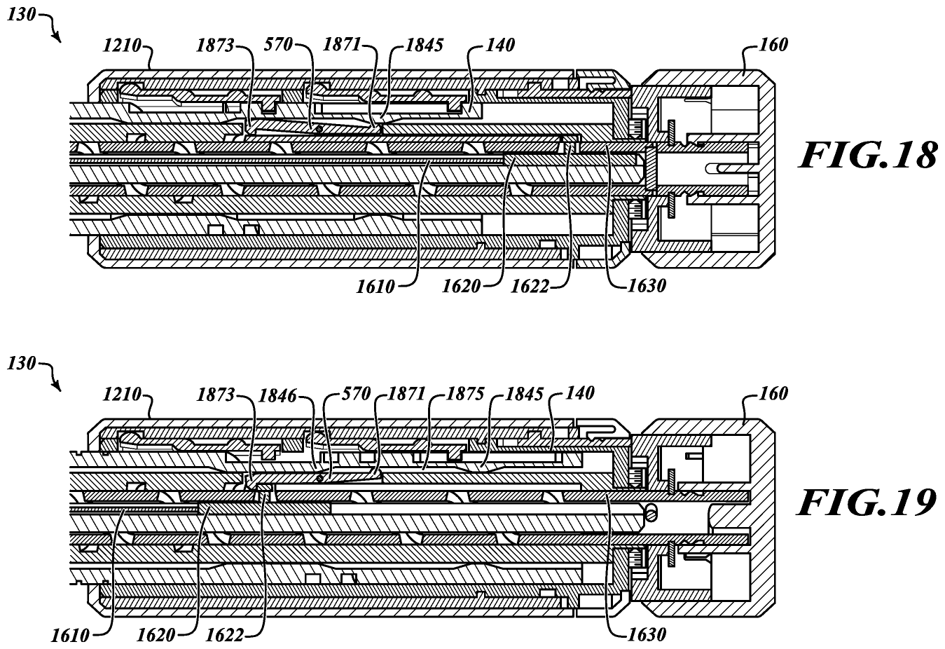

The depth control mechanism 150 is selectively positionable along the housing (including the housing sections 505 and 506), and in various embodiments is rotatably advanced to set a needle penetration depth, as explained further below. The depth control mechanism 150 is configured to act as a stop for the control handle 130. The housing sections 505 and 506 also are configured to receive within the cam tube 440. The cam tube 440 supports the thrust bushing 430 that couples the needle 420 to the cam tube 440 so that the needle 420 is slidably receivable within the sheath 102. The cam tube 440 defines a plurality of helical grooves 541 to engage a stylet carriage (not shown) so that rotating the cam tube 440 by rotating the coil knob 160 advances the stylet to extend the coilable filament (neither of which are shown in FIG. 5). A travel stop 570 is selectively engaged to limit travel of the stylet carriage as further described below with reference to FIGS. 18 and 19.

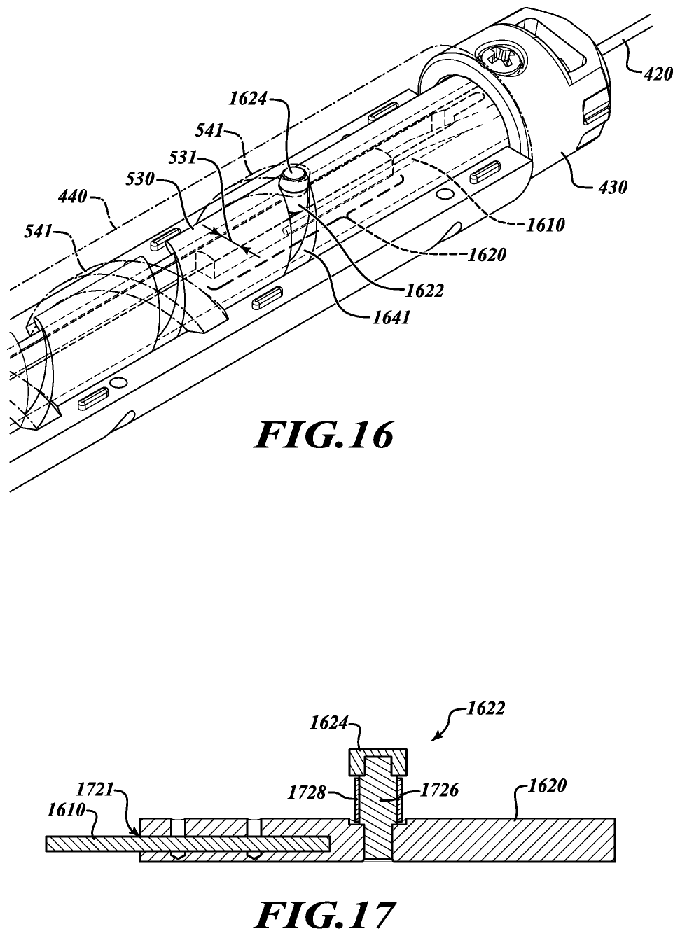

A rotatable cam shaft 530 is received within the cam tube 440. A guide slot 531 of the rotatable cam shaft 530 guides a drive member (not shown in FIG. 5) that causes the coilable filament to be extended. Engagement of the drive member extending through the guide slot 531 of the cam shaft 530 is engaged by the cam tube 440 to control extension of the coilable filament, as further described below with reference to FIGS. 16 and 17.

Referring to FIG. 6, the sheath mechanism 110 of the user interface 100 facilitates coupling of the user interface 100 with the positioning system 300 and enables an operator to control a positioning of the sheath 102. In various embodiments, the sheath mechanism 110 includes a coupling 602 in the nature of a Luer connector that is rotatable by a knurled knob 604 or other grippable structure to rotate the coupling 602 to secure the sheath mechanism 110 to the port 302 of the positioning system 300 (FIG. 3).

In various embodiments, the sheath mechanism 110 permits positioning of sheath 102 by controlling movement of the housing 104 that is coupled to the sheath 102 and that supports the mechanisms that operate the needle and the stylet used to deploy the coils, as described below. The sheath 102 thus moves with the housing 104. Moving the housing 104 to position the sheath 102 also positions the other mechanisms supported by the housing 104 to move with the sheath 102, allowing the mechanisms to operate within the frame of reference of the sheath 102. The sheath 102 is coupled with the housing 104 at the sheath hub 419, as previously described with reference to FIG. 4.

In various embodiments, the sheath lock 412 extends from the housing 104 where it is secured to the housing at the nut plate 415 (not shown in FIG. 6) as described with reference to FIG. 4 or by a similar structure. In various embodiments, the sheath lock 412 includes a knurled screw, but other securing devices may be used. The sheath lock 412 is slidably received within the channel 414 in the sheath cap 416 which is fixably joined to the coupling 602. When the sheath lock 412 is in an unlocked or loosened position, the sheath lock 412 extends away from the sheath cap 416, thereby enabling the sheath lock 412 to slide within the channel 414. As a result, when the sheath lock 412 is in an unlocked position, the housing 104 is able to slide relative to the sheath cap 416 to permit movement of the sheath 102 (as well as the enclosed needle and the other mechanisms, not shown in FIG. 6) to position the sheath 102 at a desired location, as further described below. On the other hand, when the sheath lock 412 is in a locked or tightened position, the sheath lock 412 frictionally engages the sheath cap 416 to prevent movement of the housing 104 relative to the sheath cap 416 and the coupling 402, thus, preventing movement of the sheath 102 and the housing 104.

Referring to FIGS. 7A-8B, operation of the sheath mechanism 110 for positioning the sheath 102 at a desired location near a surface 201 is described. Referring to FIG. 7A, the user interface 100 is joined to the port 302 of the positioning system 300 by the coupling 602 of at the end of the sheath cap 416. The sheath lock 412 may initially rest at a trailing edge 715 of the channel 414 before the sheath 102 is moved into a desired location. The sheath lock 412 may be unlocked or loosened to permit the sheath mechanism 110 to permit the housing 104 to be moved relative to the sheath cap 416 to extend the sheath 102 into a desired position.

Referring to FIG. 7B, a distal end 701 of the sheath 102 is shown in the vicinity of the surface 201 where it is desired to deploy coils. The sheath 102 contains the needle 420 (shown as a dashed line) which in turn contains a coilable filament 710 (shown as a dotted line). At this point in operation of the user interface 100, a distal end 703 of the needle 420 is contained within the distal end 701 of the sheath 102, and a distal end 705 of the coilable filament 710, in turn, is contained within the distal end 703 of the needle 420. As a result of the coilable filament 710 being contained within the needle 420, the coilable filament 710 is constrained into a straight, uncoiled configuration. The sheath 102 contains the needle 420 and the coilable filament 710 to protect the needle 420 and the coilable filament 710 as they are being extended into position. The sheath 102 also may serve to protects the controllable guiding apparatus 304 (FIG. 3; not shown in FIG. 7B) from possible damage that may result from contact with the needle 420 or the coilable filament 710.

Referring to FIGS. 8A and 8B, the sheath mechanism 110 is manipulated to extend the distal end 701 of the sheath 102 into position adjacent to the surface 201. As previously described, unlocking or loosening the sheath lock 412 enables the housing 104 to move within the sheath cap 416 to advance the sheath 102. As shown in FIG. 8A, the housing 104 is moved through a distance 809 toward and into the sheath cap 416 to extend the sheath 102, as represented by the sheath lock 412 moving to an intermediate point 815 of the channel 414. The sheath lock 412 may be locked or tightened when the sheath 102 is positioned at a desired location. Referring to FIG. 8B, the distal end 701 of the sheath 102 is moved through the distance 809 to a desired location at or adjacent to the surface 201. Because the housing 104 supports the needle and coil control mechanisms as previously described, advancing the housing 104 not only advances the distal end 701 of the sheath 102 toward the surface 201, but also moves the distal end 703 of the needle 420 and the distal end 705 of the coilable filament 710 toward the surface 201. As will be appreciated, and will subsequently be described, the sheath mechanism 110 also may be used to withdraw the sheath 102 from the surface 201. In various embodiments, withdrawal of the sheath 102 from the surface is performed during the process of deploying the coilable filament 710 to facilitate positioning of the coilable filament 710, as further described below.

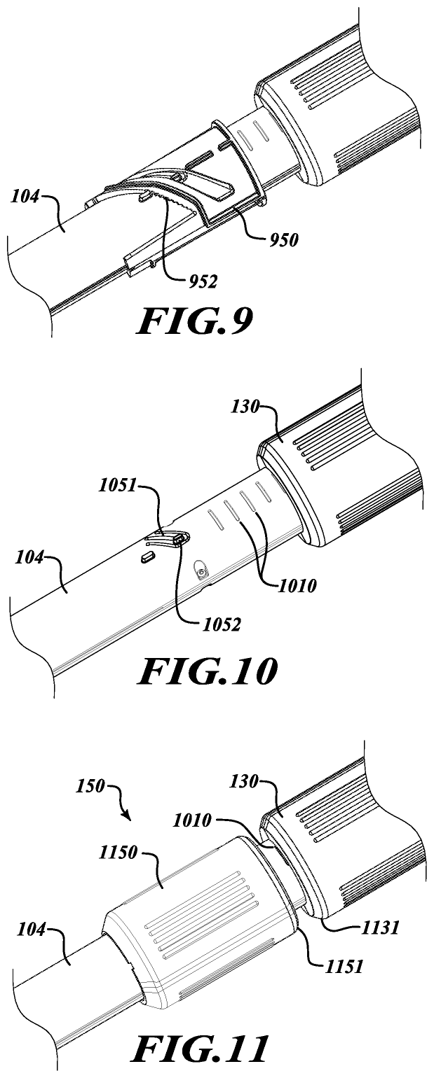

Referring to FIGS. 9-11, in various embodiments, the depth control mechanism 150 may be moved to set a depth limit for extension of the needle 420. Referring to FIG. 9, the depth control mechanism 150 may include a locking sleeve 950 that resides beneath a depth control grip 1150 (FIG. 11). The locking sleeve 950 guides the depth control grip 1150 by guiding one or more inward-facing pins (not shown in FIG. 9) extending from the depth control grip 1150. The one or more pins and may include a number of staircase threads 952 to positively engage the one or more pins extending inwardly from the depth control grip 1150. The staircase threads 952 engaging the pin on the depth control grip 1150 provide a degree of resistance to the movement to support a user's ability to make precise settings in moving the depth control mechanism 150 to a desired position. The staircase threads 1052 may to provide tactile and/or audible feedback as the depth control grip 1150 is moved.

Referring to FIG. 10, the housing 104 may support one or more cantilever springs 1051, each of which may support an outward-facing movable pin 1052 to engage an underside of the depth control grip 1150 (not shown in FIG. 10). Force exerted by the pin 1052 on the cantilever spring 1051 may hold the depth control grip 1150 in place once the depth control grip 1150 is moved to a desirable position along the housing 104. As also shown in FIG. 10, for user guidance, the housing 104 may include molded depth markers 1010 to assist the user in setting the depth control mechanism 150 to a desired depth.

Referring to FIG. 11, the depth control grip 1150 is rotatable about the housing 104 to position the depth control mechanism 150 at the desired position. Once set at the desired position, optionally with reference to the depth markers 1010, a trailing edge 1151 of the depth control grip 1150 is positioned to abut a leading edge 1131 of the control handle 130. Thus, as described further below, when the control handle 130 is slidably advanced along the housing to extend the needle (not shown) to pierce a surface, the depth control mechanism 150 stops the control handle 130 from further extending the needle beyond the desired depth set with use of the depth control mechanism 150.

Referring to FIG. 12, the control handle 130 interoperates with a number of components to facilitate and control movements of the control handle in moving the needle 420 and/or the coilable filament 710. A control grip 1210 presents the user interface of the control handle 130. Movements of the control grip 1210 are managed by an inner control sleeve 1240 that selectively engages the control channels 934 on the housing 104 via other components described below. According to various embodiments, operation of the control grip 1210 is constrained to operate only in an appropriate sequence, predetermined directions, and within a predetermined range to ensure that the extension and retraction of the needle 420 and positioning of the coilable filament 710 are desirably accomplished.

The inner control sleeve 1240 supports pin rockers 1241 and 1242 rotatably disposed in openings 1251 on the inner control sleeve 1240. The pin rockers 1241 and 1242, which are further described below, are rotatably mounted at a mid-point and support outward-facing protrusions that are selectively engaged by the planar inner surface 1243 of the cam plate 1244 or received by recesses 1247-1249 formed in the inner surface of a cam plate 1244. The recesses 1247-1249 are shaped so that movements of the control grip 1210 result in the recesses 1247-1249 in the cam plate 1244 rotating the pin rockers 1241 and 1242 to cause inward-facing pins to selectively engage the control channels 532 defined by the housing 104 to direct movement of the control handle 160 to selectively move the needle 420 and the coilable filament 710. It will be appreciated that lateral edges of the recesses 1247-1249 are sloped so that rotational movement of the cam plate 1244 slidably engage the outward-facing protrusions to rotate the pin rockers 1241 and 1242.

The cam plate 1244 may be joined with a detent plate 1249 to form a sleeve. The detent plate 1249 may include a cantilever spring arm 1253 to engage detents or protrusions (not shown) on the inner control sleeve 1240 to provide tactile resistance to facilitate controlled movements of the control grip 1210 in directing operations of the control handle 130. The control grip 1210 is fixably mounted over the cam plate 1244 and the detent plate 1249 so that translational and rotational movement of the control grip 1210 results in corresponding translational and rotational movement of the cam plate 1244 and the detent plate 1249.

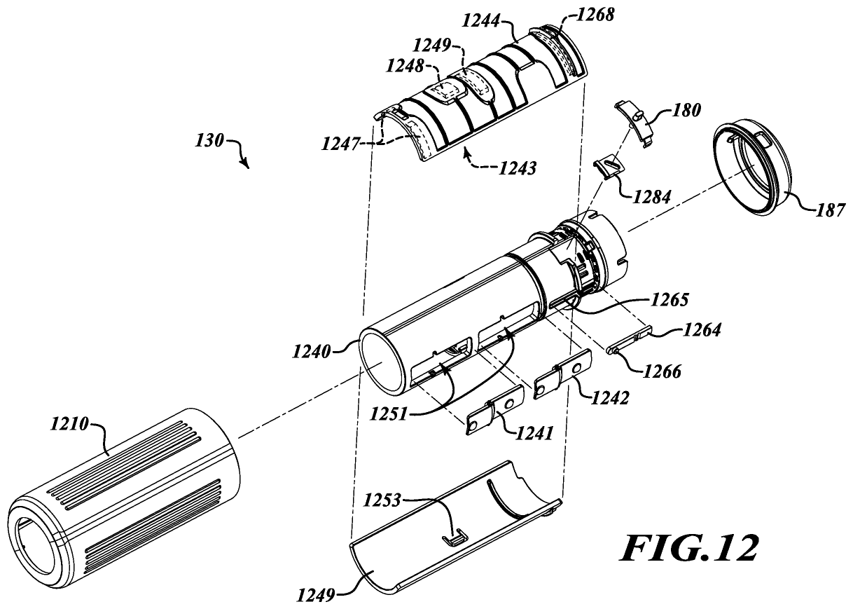

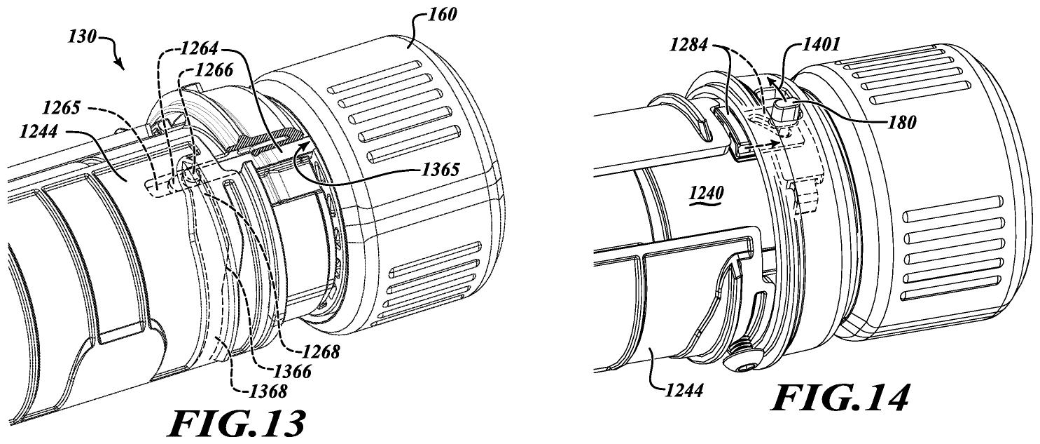

Continuing to refer to FIG. 12, components of the interlock mechanism also include a locking bar 1264 and the retraction lock 180. The locking bar 1264 is slidably mounted in a channel 1265 on the inner control sleeve 1240 and supports a protrusion 1266 that is engaged by a locking recess 1268 on an inner surface of the cam plate 1244, as further described below with reference to FIG. 13. The retraction lock 180 is configured to prevent rotation of the camp plate 1244 and, thus, prevent the control grip 1210 from being moved to withdraw the needle 420 unless the retraction lock 180 is specifically unlocked by the user, as described further below. Operation of the release button crank is further described below with reference to FIG. 14.

Referring to FIG. 13, the locking bar 1264 is in a locked position, extending from the channel 1265 of the inner control sleeve 1240 into a recess 1365 of the coil knob 160. As the control grip 1210 (not shown in FIG. 13) is rotated to rotate the cam plate 1244, the locking recess 1268 rotates across the protrusion 1266. A ramped portion 1366 of the locking recess 1268 laterally engages the protrusion 1266 and drives the protrusion into an unlocking slot 1368 of the locking recess 1268. Driving the protrusion 1266 is driven into the unlocking slot 1368 slides the locking bar 1264 across the channel 1265 and withdraws the locking bar 1264 from engagement with the coil knob 160. At this point, the coil knob 160 may be rotated to extend the coilable filament 710 (not shown in FIG. 13).

Referring to FIG. 14, the release lock 1284 is in a locked position, to block rotation of the cam plate 1244 to permit withdrawal of the needle 420. To permit withdrawal of the needle 420, a user will slide the retraction lock 180 laterally. Sliding the retraction lock 180 in a direction represented by arrow 1401 allows the cam plate 1244 and the control grip 1210 to be rotated to permit withdrawal of the needle 420.

Referring again to FIG. 12, the recesses 1247-1249 in the cam plate 1244 may manipulate the pin rockers 1241 and 1242 to control movement of the control handle 130. Referring back to FIG. 5, the housing 104 provides control channels 532, including one or more channels 534 extend along an axis of the housing 104 while one or more channels 536 extend radially around the housing 104. The control channels 532 thus may selectively allow for either linear or rotational motion constrained by control handle when pins extending from the pin rockers 1241 and 1242 extend through the openings 1251 in the inner control sleeve 1240 to engage the control channels 532. The interaction between the cam plate 1244 and the pin rockers 1241 and 1242 controls which pins engage which of the control channels 532. The pin rockers 1241 and 1242, as manipulated by the recesses 1247-1249, selectively retract and engage protrusions from the pin rockers 1241 and 1242 into selected control channels 532 so that the control handle 160 is constrained to move in a specified manner consistent with the successive steps of positioning the needle 420 and the coilable filament 710.

To illustrate, referring to FIG. 15, the cam plate 1244 is configured so that the planar inner surface 1243 of the cam plate 1244 and the recesses 1247-1249 formed in the cam plate 1244 selectively direct pins extending from the pin rollers 1241 and 1242 into selected control channels 532 defined by the housing 104 (not shown in FIG. 15). The housing 104 is not shown in FIG. 15, but as will be appreciated, would extend directly beneath the inner control sleeve 1240.

For example, the pin roller 1241 has an inwardly-extending pin 1551 and two outwardly-extending protrusions 1548 and 1552 and the pin roller 1242 has an inwardly-extending pin 1561 and two outwardly-extending protrusions 1557 and 1559. With the control grip 1210 rotating the cam plate 1244 into its current position, the planar inner surface 1243 of the cam plate 1244 presses against the protrusion 1548 to rotate the pin rocker 1241 in a counterclockwise direction, while the recess 1248 in the cam plate 1244 allows the protrusion 1552 to rotate away from the housing 104 to lift the pin 1551 away from the housing 104. Accordingly, the pin 1551 is withdrawn from any control channels 532 in the housing 104, and the pin 1551 does not constrain or direct movement of the control handle 130.

At the same time, however, the planar inner surface 1243 of the cam plate 1244 presses against the protrusion 1559 to rotate the pin rocker 1242 in a clockwise direction, while the recess 1249 in the cam plate allows the protrusion 1557 to rotate away from the housing 104. As a result, the pin 1561 is driven downwardly into the control channel 532. Thus, the pin 1561 and the shape of the control channel 532 receiving the pin 1561 will direct and constrain the movement of the control handle 130. Thus, if the control channel 532 engaged by the pin 1561 is aligned axially with the housing 104, the control handle may slide along that control channel 532 and/or if the control channel 532 is aligned radially along the housing 104, the control handle 130 may be rotated as directed by the control channel 532. Thus, the shape of the recesses in the cam plate 1244 and the shape of the control channels 532 may be used to direct movements of the control handle 130.

Referring to FIG. 16, the coilable filament 710 (not shown in FIG. 16) is motivated by a stylet 1620 that extends through the needle 420. The coilable filament 710, as further described below, is releasably coupled with a distal end of the stylet 1620. The stylet 1610 is coupled with a stylet carriage 1620 that is slidably received within the guide slot 531 of a cam shaft 530. The stylet carriage 1620 supports a drive member 1622, extending radially from the stylet carriage 1620. In various embodiments, the drive member 1622 supports a cap roller 1624 that is rotatably attached to an end of the drive member 1620. The drive member 1622 engages an inner surface of the helical channels 541 of the cam tube 440, as highlighted by a projected helical channel 1641. As a result of the engagement of the drive member 1622 between the helical channels 541 of the cam tube 440 and the guide slot 531 of the cam tube 530, as the cam tube 440 is rotated by an operator turning the coil knob 160 (not shown in FIG. 16), the scissoring-type interaction between the guide slot 531 of the cam shaft 530 and the helical channels 541 of the cam tube 440 impart a translational force to the drive member 1620 along an axis of the stylet 1610. Thus, rotation of the cam tube 440 pushes the stylet carriage 1620 through the cam tube 1630 to advance the stylet 1610 and to extend the coilable filament 710 as further described below.

The guide channel 1632 of the cam shaft 530 also provides lateral support to the stylet 1610. As a result, as the stylet carriage 1620 is driven through the cam shaft 530 and the stylet 1610 encounters resistance, the guide channel 1632 supports sides of the stylet 1610 to prevent the stylet from buckling.

Referring to FIG. 17, the stylet carriage 1620 is fixably coupled to the stylet 1610. The stylet 1610 may be partially received within the stylet carriage 1620 in recess 1721. The stylet 1610 may be secured to the stylet carriage 1620 by bonding. The drive member 1622 in various embodiments includes a drive pin 1726 fixably coupled to the stylet carriage 1620. The cap roller 1624 may be mounted on the drive pin via a sleeve 1728 to facilitate the rolling of the cap roller 1624 as the cap roller engages the helical channel 541 of the cam tube 440.

Referring to FIGS. 18 and 19, the travel stop 570 is positionable to selectively arrest movement of the stylet carriage 1620. By arresting movement of the stylet carriage 1620, the extension of the stylet 1610 is limited to control extension of the coilable filament 710 (not shown in FIGS. 18 and 19) beyond a desired point. Referring to FIG. 18, in a mode where the needle 420 is retracted from the surface 201 (neither of which is shown in FIG. 18 or 19), as described further below, there is no need to restrict movement of the stylet 1610 during extension of the needle 420 or to limit extension of the coilable filament 710 as a second segment of the coil is deployed. Accordingly, the travel stop 570 is manipulated to not interfere with movement of the stylet carriage 1620. A first cam lobe 1845 extending from the housing 140 engages a protrusion 1871 extending outwardly from the travel stop 570. The engagement of the first cam lobe 1845 with the protrusion 1871 causes the travel stop to rotate in a clockwise direction, lifting a stop 1873 away from the cam shaft 530 and, thus, out of the path of the stylet carriage 1620. As a result, the travel stop 570 does not inhibit movement of the stylet carriage 1620.

However, when the needle 420 is extended through the surface 201, it is desirable to limit travel of the stylet carriage 1620 so that the stylet 1610 advances only a desired length of the coilable filament 710 beyond the end of the needle 420, leaving a desired portion of the coilable filament 710 to be deployed on the proximal side of the surface 201 after withdrawal of the needle 420. The travel stop 570 permits control over the travel of the stylet carriage 1620 to limit the extension of the stylet 1610 and, thus, the coilable filament 710. Referring to FIG. 19, with the needle 420 extended, a second cam lobe 1846 engages the travel stop 570 to rotate the travel stop 570 in a counterclockwise direction. A recess 1875 defined by the housing 140 permits the protrusion 1871 to move away from the cam shaft 530. The stop 1873 is thus moved toward the cam shaft 530 and into the path of the drive member 1622 extending from the stylet carriage 1620 to arrest movement of the stylet carriage 1620. Accordingly, movement of the stylet carriage 1620 is controlled to prevent the stylet 1610 from being advanced to extend too much of the coilable filament 710 when the needle 420 is extended beyond the surface 201.

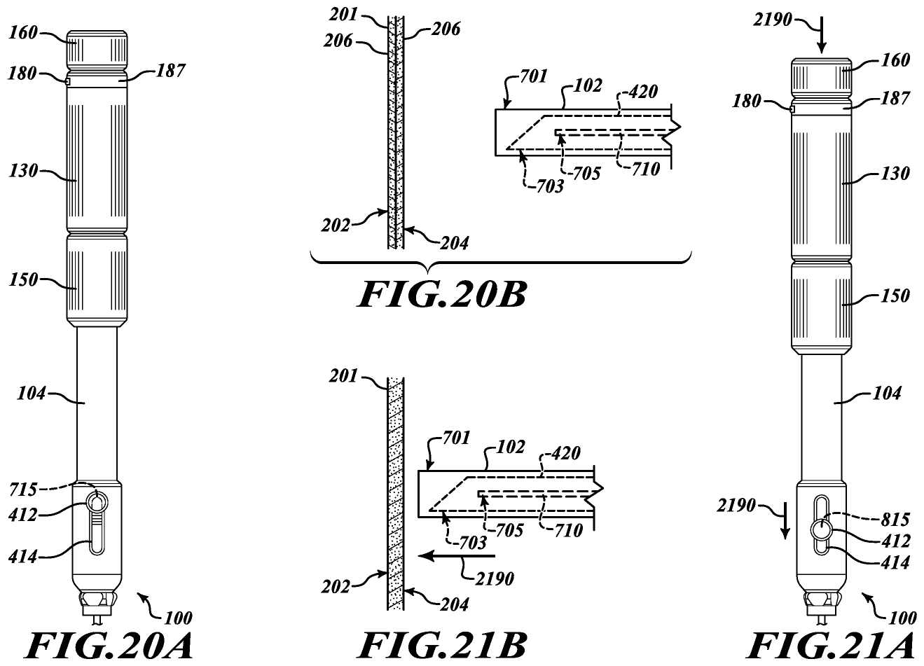

Operation of the user interface 100 and the resulting action in extending the sheath 102, the needle 420, and the coilable filament 710 are explained with reference to FIGS. 20A-31B. In the first of each of the pair of figures, for example in FIG. 20A, the state of the user interface 100 is shown, while in the second of each of the pair of figures, for example in FIG. 20B, the effect at the distal ends of the sheath 102, the needle 520, and/or the coilable filament 710 are shown. Components of the user interface 100 involved in any of the steps in the deploying of the coilable filament are labelled in each of FIGS. 20A, 21A, 22A, 23A, 24A, 25A, 26A, 27A, 28A, 29A, 30A, and 31A. Positions of the distal end 701 of the sheath 102, the distal end 703 of the needle 420, and the distal end 705 of the coilable filament 710 relative to the surface 201 where the first coil 212 and the second coil 214 are to be deployed on the distal side 202 and the proximal side 204 of the surface 201, respectively, are shown in each of FIGS. 20B, 21B, 22B, 23B, 24B, 25B, 26B, 27B, 28B, 29B, 30B, and 31B.

In the examples shown in FIGS. 20A-31B, the surface 201 where the first coil 212 and the second coil 214 are to be deployed may be regarded without limitation as adjacent portions of a common bile duct wall 208 and a small intestine wall 206. However, it will be understood that the user interface 100 may be used for the deployment of coils on any type of surface 201 that may be pierceable by the needle 607. It also will be appreciated that, as described with reference to FIG. 18, the coilable filament 611 is driven through the needle by the 1863 stylet, although the views of the surface 201 shown in FIGS. 20B, 21B, 22B, 23B, 24B, 25B, 26B, 27B, 28B, and 29B show only the coilable filament 611.

It will be appreciated that, the following operational description of the user interface 100 described with reference to FIGS. 20A-31B focus on the operation of control surfaces, such as the control handle 130, and any resulting movement of the sheath 102, the needle 420, and the coilable filament 710. As will be appreciated, movement of any of the control surfaces results in movement of other components. For example, rotation of the control handle 130 may involve movement of associated components such as the cam plate 1244 and the pin rockers 1241 and 1242 as previously described. In the interest of clarity and brevity, the movement of each component resulting from movement of a control surface. Similarly, in describing movements of the distal end 701 of the sheath 102, the distal end 703 of the needle 420, and the distal end 705 of the coilable filament 710 necessarily involve movement of the mechanisms attached thereto as previously described. For example, advancing the distal end 705 of the when the coilable filament 710 necessarily involves movement of the stylet 1610, the stylet carriage 1620, and other components as previously described. Again, in the interest of clarity and brevity, the movement of each component that result in movement of the sheath 102, the needle 420, and the coilable filament 710 are not repeated here.

Referring to FIGS. 20A and 20B, the user interface 100 is in a starting position when the user interface 100 has been coupled with the positioning system 300 (FIG. 3) and the positioning system 300 has been used to convey the distal end 701 of the sheath 102 to a position adjacent to the surface 201 where deployment of the coilable filament 710 is to occur. As shown in FIG. 20A, and as previously shown in FIG. 6A, the sheath lock 412 is situated in a starting position at a trailing edge 715 of the channel 414. Referring to FIG. 20B, the distal end 701 of the sheath 102 is positioned near the surface 201. The distal end 703 of the needle 420 rests within the distal end 701 of the sheath 102, and the distal end 705 of the coilable filament 710 rests within the distal end 703 of the needle 420 at a proximal side 204 of the surface 201.

Referring to FIGS. 21A and 21B, the distal end 701 of the sheath 102 is extended toward the surface 201. As previously described with reference to FIGS. 8A and 8B, the sheath lock 412 is unlocked or loosened and moved a distance 2190 to an intermediate point 815 in the channel 414 to advance the distal end 701 of the sheath 102. The distal end 705 of the coilable filament 710 and the distal end 703 of the needle 420 slide in concerted motion with the distal end 701 of the sheath 102. Accordingly, the components of the user interface 100 that are coupled with the needle 420 and the coilable filament 710, including the coil knob 160, the control handle 160, the needle depth control mechanism 150, and the housing 104 also move through the distance 2190. The movement of the components of the user interface 100 result in a corresponding movement of each of the distal end 701 of the sheath 102, the distal end 703 of the needle 420, and the distal end 705 of the coilable filament 710 through the same distance 2190.

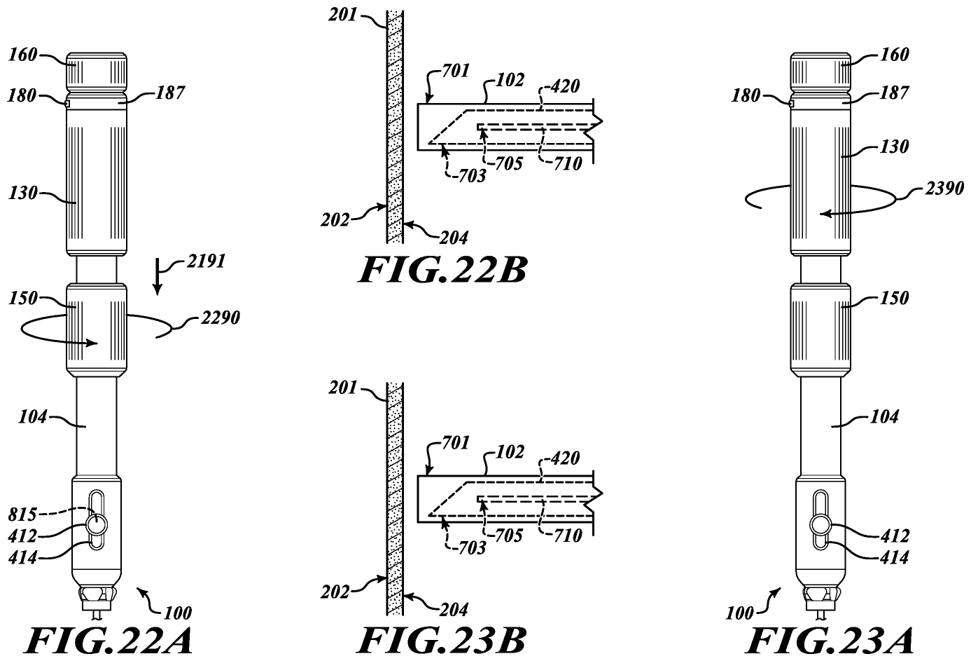

Referring to FIGS. 22A and 22B, the user interface 100 is being prepared for extension of the needle 420 by rotating the needle depth control mechanism 150 in a direction 2290 to move the needle depth control mechanism 150 through a distance 2291 to a desired location. As previously described with reference to FIGS. 9-11, the needle depth control mechanism 150 rotatably engages depth setting channels 952 on the housing 104 to set the position of the needle depth control mechanism 150 and, thus, to set a stop to limit travel of the control handle 130. The rotation of the needle depth control mechanism 150 to move the needle control mechanism 150 through the distance 2291 along the housing 104 permits subsequent movement of the control handle 130 through a same distance 2291. During the movement of the needle depth control mechanism 150, other components of the user interface 100 remain at a same position, including the control handle 130, the coil knob 160, and the sheath lock 412. Referring to FIG. 22B, because this portion of the process only involves preparing for movement of the needle 420, the distal end 701 of the sheath 102, the distal end 703 of the needle 420, and the distal end 705 of the coilable filament 710 remain in a same position as in FIG. 21B.

Referring to FIGS. 23A and 23B, the user interface 100 is being further prepared for extension of the needle 420 by unlocking the control handle 130. The control handle 130 is unlocked by rotating the control handle 130 in a direction 2390 relative to the housing 104 and other components. As previously described with reference to FIGS. 12 and 15, the rotation of the control grip 121—of the control handle 130 results in movement of the cam plate 1244 and the pin rockers 1241 and 1242 to configure the control handle 130 to engage control channels 532 on the housing 104 for extension of the needle 420. During the rotation of the control handle 130, other components of the user interface 100 remain at a same position, including the housing 104, the coil knob 160, the depth control mechanism 150, and the sheath lock 412. Referring to FIG. 23B, because this portion of the process only involves preparing for movement of the needle 420, the distal end 701 of the sheath 102, the distal end 703 of the needle 420, and the distal end 705 of the coilable filament 710 remain in a same position as in FIG. 22B.

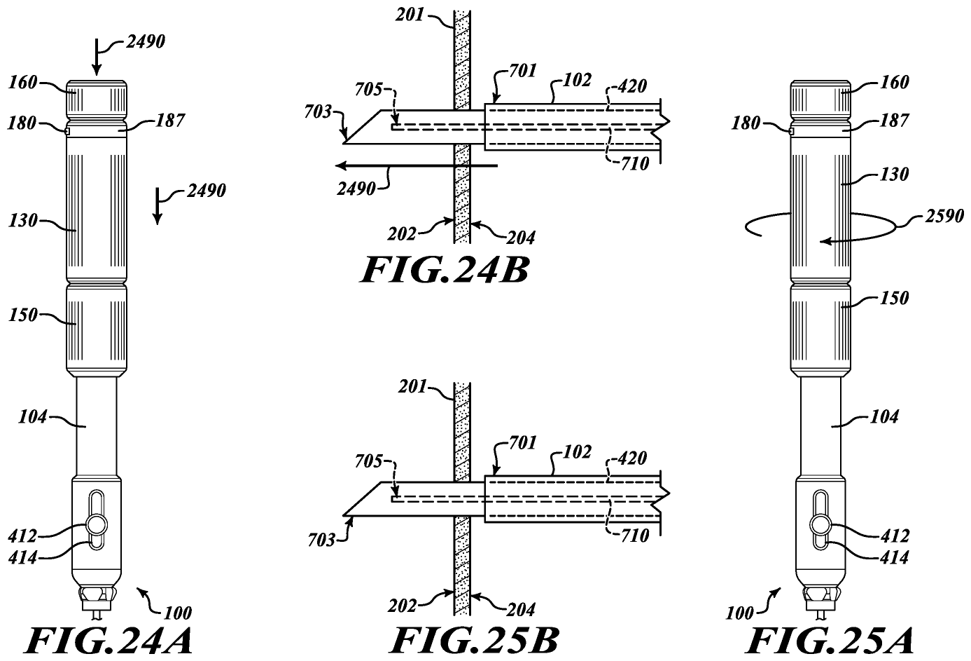

Referring to FIGS. 24A and 24B, the user interface 100 is manipulated to extend the distal end 703 of the needle 420 to pierce the surface 201 in preparation for deployment of the first coil 212 (FIGS. 2A and 2B). The control handle 130 is moved through a distance 2490. A user may slide the control handle 130 slides until it abuts the needle depth setting mechanism 150, stopping the extension of the needle at the depth set using the needle depth setting mechanism 150 as described with reference to FIGS. 22A and 22B. Because the coilable filament 710 moves with the needle 420 to prepare for deployment of the coilable filament 710, the coil knob 160 moves in concert with the control handle 130, along with other components such as the retraction lock 180 and the interlock indicator 187. Referring to FIG. 24B, the movement of the control handle 130 and other components through the distance 2490 results in extension of the distal end 703 of the needle 420 and the distal end 705 of the coilable filament 710 through the distance 2490. The distal end 703 of the needle 420 thus pierces the surface 201, thereby passing from the proximal side 204 of the surface 201 to the distal side 202 of the surface 201. The needle 420 is thus in position for extension of the coilable filament 710 to deploy the first coil 212, as is further described below.

Referring to FIGS. 25A and 25B, the user interface 100 is manipulated to prepare for extension of the coilable filament 710. To prepare for extension of the coilable filament 710, the control handle 130 is rotated in direction 2590 to unlock the coil knob 160 so that it can be rotated to advance the stylet 1610 (not shown in FIGS. 25A and 25B) to advance the coilable filament 710 as previously described with reference to FIGS. 12 and 13. Referring to FIG. 25B, because this portion of the process only involves unlocking the coil knob 160 to prepare for advancement of the stylet 1610 and the coilable filament 710, the distal end 701 of the sheath 102, the distal end 703 of the needle 420, and the distal end 705 of the coilable filament 710 remain in a same position as in FIG. 24B.

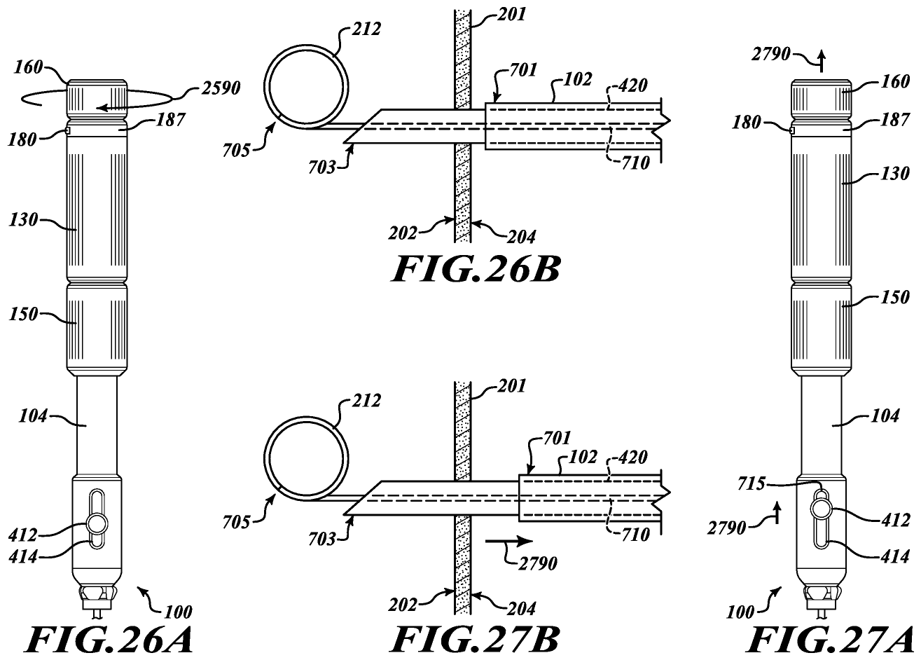

Referring to FIGS. 26A and 26B, the user interface 100 is manipulated to advance the stylet 1610 (not shown in FIGS. 26A and 26B) to extend the coilable filament 710. Specifically, the coil knob 160 is rotated in a direction 2690 to extend the coilable filament 710. As previously described, rotating the coil knob 160 rotates the cam tube 440, thereby causing the helical channels 541 of the cam tube 440 to advance the carriage stylet 1620 to advance the stylet 1610, in turn extending the coilable filament 611. During the rotation of the coil knob, other components of the user interface 100 remain at a same position, including the control handle 130 and other components. Referring to FIG. 26B, the distal end 705 of the coilable filament 710 is extended beyond the distal end 703 of the needle 420. As previously described, in various embodiments, the coilable filament 710 is formed of a memory wire configured to coil when the needle 420 no longer constrains the coilable filament 420 to remain in a straightened configuration. Thus, extension of the coilable filament 710 beyond the distal end 703 of the needle 420 automatically results in the formation of the first coil 212. During the deployment of the first coil 212, the distal end 701 of the sheath 102 and the distal end 703 of the needle 420 remain in a same position as in FIG. 25B.

Referring to FIGS. 27A and 27B, to prepare for withdrawal of the needle 420 from the distal side 202 to the proximal side 204 of the surface 201 for deployment of the second coil 214, the sheath 102 is partially retracted from the surface 201. To retract the sheath 102, the sheath lock 412 is released and the housing 104 is moved in a direction 2790 to retract the distal end 701 of the sheath 102. Referring to FIG. 27B, the movement of the sheath lock 412 through the distance 2790 results in a corresponding movement of the distal end 701 of the sheath 102 through a distance 2790 to retract the distal end 701 of the sheath 102 away from the proximal side 204 of the surface 201.

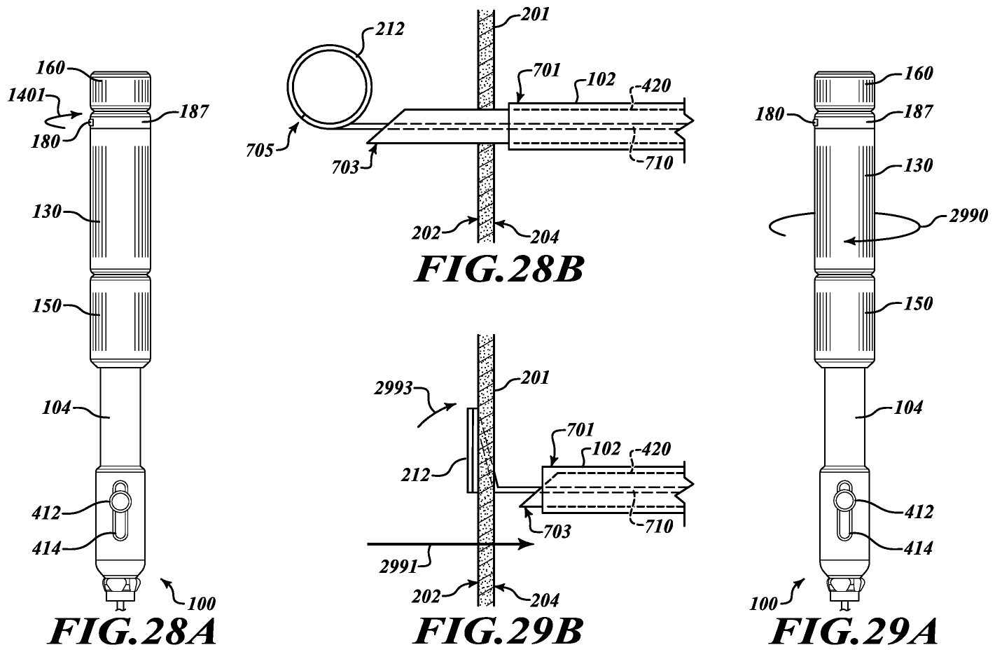

Referring to FIGS. 28A and 28B, retraction of the needle 420 in preparation for the deployment of the second coil 214 is initiated by rotating the retraction lock 180 as described with reference to FIG. 14. Sliding the retraction lock 140 in the direction 1401 rotates the release button crank 1284 (not shown in FIG. 14) to enable the cam plate 1244 and, thus, the control handle 130 to be rotated to permit withdrawal of the needle 420. Referring to FIG. 28B, because this portion of the process only involves actuating the retraction lock 180 to prepare for withdrawal of the needle 420, the distal end 701 of the sheath 102, the distal end 703 of the needle 420, and the distal end 705 of the coilable filament 710 remain the same positions as in FIG. 27B.

Referring to FIGS. 29A and 29B, the user interface 100 is manipulated to retract the needle 207 by rotating the control handle in a direction 2990. Rotating the control handle 130 rotatably withdraws the needle 420 from the surface 201. At the same time, rotating the control handle 130 rotates the cam tube 440 and thus the rest of the components coupled with the stylet 1610 and, thus, the coilable filament 710. Referring to FIG. 29B, the rotation of the control handle 130 to rotatably withdraw the needle 420 retracts the distal end 703 of the needle 420 by a distance of 2991. At the same time, however, rotation of the coilable filament 710 causes the coilable filament 710 and, thus, the first coil 212 to twist in a direction 2993, flipping the first coil 212 to lie against the distal side 202 of the surface 201.

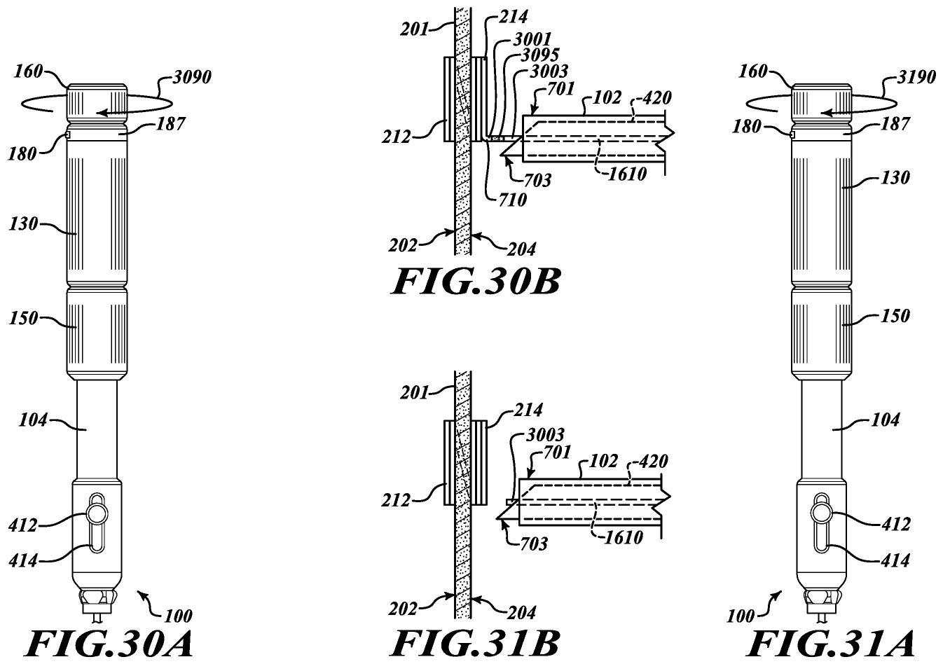

Referring to FIG. 30A, the user interface 100 is manipulated to deploy the second coil 214. To deploy the second coil 214, after retraction of the needle 420 to the proximal side 204 of the surface 201 and the concurrent twisting of the coilable filament 710 to flip the orientation of the first coil 212, the coil knob 160 is further rotated in a direction 3090 to further advance the stylet 1610 (not shown in FIG. 30A). Referring to FIG. 30B, an additional length of the coilable filament 710 extends beyond the distal end 703 of the needle 420, resulting in deployment of the second coil 214 on the proximal side 204 of the surface 201. Without further rotating of the coilable filament 710, the additional length of the coilable filament 710 is deployed in a same orientation of the first coil 212. Thus, the second coil 214 lies along the proximal side 204 of the surface 201, parallel with the first coil 212.

Continuing to refer to FIG. 30B, the rotation of the coil knob 160 advances the stylet 1610 such that both a proximal end 3001 of the coilable filament 710 and a distal end 3003 of the stylet 1610 near the distal end 703 of the needle 420. In various embodiments, both the proximal end 3001 of the coilable filament 710 is joined with the distal end 3003 of the stylet 1610 with a releasable weld 3095. The releasable weld 3095 is configured to hold the coilable filament 710 to the stylet 610 as both translate through the needle 420. However, as described below, the releasable weld 3095 is not configured to withstand the torque to be applied to the releasable weld caused by the twisting of the coilable filament 710 beyond the distal end 703 of the needle 420 resulting from the twisting of the coilable filament 710 to deploy the coil 212 and 214 as previously described.

Referring to FIGS. 31A and 31B, the user interface 100 is manipulated to complete the deployment of the second coil 214 and to disengage the second coil 214 from the stylet 1610. The coil knob 160 is rotated in a direction 3190 drives to advance the distal end 3003 of the stylet 1610 to and/or slightly beyond the distal end 703 of the needle 420. As previously described, in various embodiments, without the support of the needle 420, the releasable weld 3095 gives way to release the coilable filament 710 from the distal end 3003 of the stylet 1610. Referring to FIG. 31B, the first coil 212 and the second coil 214 are thus left in place on the distal side 202 and the proximal side 204, respectively, on the surface 201. The needle 420, the stylet 1610, and the sheath 102 may then be retracted.

As previously described with reference to FIGS. 2A-2C, after the coils 212 and 214 have served their purpose, they may fall away. Thus, as in the example of forming a new common bile duct, after the new duct is formed, the coils 212 and 214 may fall into the small intestine to be expelled in due course from the body.

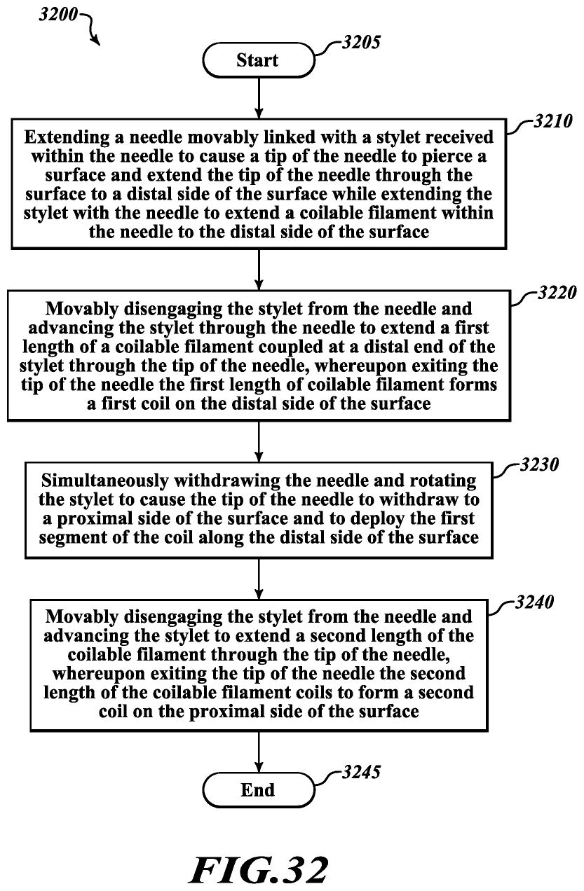

Referring to FIG. 32, an illustrative method 3200 of deploying coils by extending a coilable filament as previously described with reference to FIGS. 20A-31B is provided. The method 3200 starts at a block 3205. At a block 3210, a needle movably linked with a stylet received within the needle is extended to cause a tip of the needle to pierce a surface and extend the tip of the needle through the surface to a distal side of the surface while extending the stylet with the needle to extend a coilable filament within the needle to the distal side of the surface, as described with reference to FIGS. 24A and 24B. At a block 3220, the stylet is movably disengaged from the needle and advanced through the needle to extend a first length of a coilable filament coupled at a distal end of the stylet through the tip of the needle, whereupon exiting the tip of the needle the first length of coilable filament forms a first coil on the distal side of the surface, as described with reference to FIGS. 26A and 26B. At a block 3230, simultaneously the needle is retracted and the stylet is rotated to cause the tip of the needle to withdraw to a proximal side of the surface and to deploy the first segment of the coil along the distal side of the surface, as previously described with reference to FIGS. 29A and 29B. At a block 3240, the stylet is movably disengaged from the needle and advanced to extend a second length of the coilable filament through the tip of the needle, whereupon exiting the tip of the needle the second length of the coilable filament coils to form a second coil on the proximal side of the surface, as previously described with reference to FIGS. 30A and 30B. The method 3200 ends at a block 3245, with the coils now positioned on opposing sides of the surface.

It will be appreciated that the detailed description set forth above is merely illustrative in nature and variations that do not depart from the gist and/or spirit of the claimed subject matter are intended to be within the scope of the claims. Such variations are not to be regarded as a departure from the spirit and scope of the claimed subject matter.

What is claimed is:

1. An apparatus comprising:
a needle mechanism configured to extend a needle through a surface to a distal side of the surface and to withdraw the needle to a proximal side of the surface;
a coil mechanism configured to advance a stylet through the needle to extend a coilable filament releasably coupled at a distal end of the stylet through the needle, whereupon exiting the needle the coilable filament forms a coil; and
an interlock mechanism configured to control operations of the needle mechanism and the coil mechanism such that:
the needle mechanism and the coil control mechanism are movable to advance the needle and the stylet in concert to the distal side of the surface;
the coil mechanism is movable to advance the stylet to extend a first segment of the coil on the distal side of the surface;
the needle mechanism and the coil mechanism are movable to withdraw the needle to the proximal side of the surface; and
the coil mechanism is rotatable to further advance the stylet to deploy a second segment of the coil along the proximal side of the surface.

2. The apparatus of claim 1, wherein the needle mechanism and the coil mechanism being movable to withdraw the needle to the proximal side of the surface includes rotating the coil mechanism to deploy the first segment of the coil along the distal side of the surface.

3. The apparatus of claim 2, further comprising a control handle operably coupled with the needle mechanism, the coil mechanism and the interlock mechanism, wherein the control handle is at least one of rotatable and slidable to unlock the needle control mechanism, advance the needle mechanism and the coil mechanism to extend the needle and the coil, unlock the coil mechanism, and withdraw the needle to the proximal side of the surface.

4. The apparatus of claim 3, further comprising a coil knob coupled with the coil mechanism and rotatable to advance the stylet through the needle to extend the coilable filament.

5. The apparatus of claim 4, wherein:
the control handle is rotatable to unlock the needle mechanism;
the control handle is slidable to motivate the needle mechanism to extend the needle and the coilable filament to the distal side of the surface;
the coil knob is rotatable to advance the stylet to extend the first segment of the coil beyond the needle on the distal side of the surface;
the control handle is rotatable to withdraw the needle to the proximal side of the surface while rotating the coil mechanism to rotate the first segment of the coil to cause the first segment of the coil to lie against the distal side of the surface; and
the coil knob is rotatable to advance the stylet to apply the second segment of the coil along the proximal side of the surface.

6. The apparatus of claim 5, further comprising a depth control mechanism settable to limit travel of the needle mechanism to a settable depth, wherein the depth control mechanism is positionable to block sliding of the control handle to extend the needle when the needle has reached the settable depth.

7. The apparatus of claim 5, wherein the coil mechanism further includes:
a stylet carriage coupled with a proximal end of the stylet and supporting an outwardly extending drive member;
a cam shaft configured to slidably receive the stylet carriage and defining a guide slot to movably receive the drive member; and
a cam tube selectively couplable with the control handle and coupled with the coil knob and defining a helical channel configured to engage the drive member extending through the guide slot of cam shaft from the stylet carriage, wherein rotation of the coil knob rotates the cam shaft so that engagement of the drive member with the guide slot and the helical channel drive the stylet carriage through the cam shaft to extend the stylet as the cam shaft and is rotated relative to the cam tube.

8. The apparatus of claim 7, wherein the cam shaft is further configured to laterally support the stylet as the stylet carriage is driven to extend the stylet.

9. The apparatus of claim 5, further comprising a retraction lock that is configured to prevent the needle control mechanism from withdrawing the needle unless the retraction lock is released.

10. The apparatus of claim 5, further comprising a sheath mechanism to control a position of a sheath configured to convey the needle through a positioning system to the proximal side of the surface, wherein the sheath mechanism is configured to enable slidable movement of the sheath prior to extension of the needle through the surface and away from the proximal side of the surface prior to withdrawal of the needle to the proximal side of the surface.

11. The apparatus of claim 1, wherein the coilable filament is attached to a distal end of the stylet at a severable joint configured to disengage from the stylet once the coilable filament extends completely beyond the needle.

12. An apparatus comprising:
a housing defining a central chamber;
a sheath mechanism configured to enable slidable movement of a sheath housing a needle to a surface and to withdraw the needle from the surface;
a needle mechanism having a control handle rotatable and slidable along the housing to extend the needle and a coilable filament within the needle through the surface to a distal side of the surface and to withdraw the needle to a proximal side of the surface;
a depth control mechanism configured to be lockably positioned along the housing to limit travel of the control handle to limit travel of the needle;
a coil mechanism having a coil knob disposed at a proximal end of the housing and rotatable to advance a stylet through the needle to extend the coilable filament releasably coupled at a distal end of the stylet through the needle, whereupon exiting the needle the coilable filament forms a coil; and
an interlock mechanism coupled with the needle mechanism and the coil mechanism such that:
the needle mechanism and the coil control mechanism are movable to advance the needle and the stylet in concert to the distal side of the surface;
the coil mechanism is movable to advance the stylet to extend a first segment of the coil on the distal side of the surface;
the needle mechanism and the coil mechanism are movable to withdraw the needle to the proximal side of the surface while rotating the coil mechanism to deploy the first segment of the coil along the distal side of the surface; and
the coil mechanism is rotatable to further advance the stylet to deploy a second segment of the coil along the proximal side of the surface.

13. The apparatus of claim 12, wherein:
the control handle is rotatable to unlock the needle mechanism;
the control handle is slidable to extend the needle and the coilable filament to the distal side of the surface;
the coil knob is rotatable to advance the stylet to extend the first segment of the coil on the distal side of the surface;
the control handle is rotatable to withdraw the needle to the proximal side of the surface while rotating the coil mechanism to rotate the first segment of the coil to deploy the first segment of the coil to lie against the distal side of the surface; and
the coil knob is rotatable to advance the stylet to apply the second segment of the coil along the proximal side of the surface.

14. The apparatus of claim 12, wherein the coil mechanism further includes:
a stylet carriage coupled with a proximal end of the stylet and supporting an outwardly extending drive member;
a cam shaft configured to slidably receive the stylet carriage and defining a guide slot to movably receive the drive member; and
a cam tube selectively couplable with the control handle and coupled with the coil knob and defining a helical channel configured to engage the drive member extending through the guide slot of cam shaft from the stylet carriage, wherein rotation of the coil knob rotates the cam shaft so that engagement of the drive member with the guide slot and the helical channel drive the stylet carriage through the cam shaft to extend the stylet as the cam shaft and is rotated relative to the cam tube.

15. The apparatus of claim 14, wherein the cam shaft is further configured to laterally support the stylet as the stylet carriage is driven to extend the stylet.

16. The apparatus of claim 12, further comprising a retraction lock that is configured to prevent the needle control mechanism from withdrawing the needle unless the retraction lock is released.

17. The apparatus of claim 12, wherein the coilable filament is attached to a distal end of the stylet at a severable joint configured to disengage from the stylet once the coilable filament extends completely beyond the needle.

18. A method comprising:
extending a needle movably linked with a stylet received within the needle to cause a tip of the needle to pierce a surface and extend the tip of the needle through the surface to a distal side of the surface while extending the stylet with the needle to extend a coilable filament within the needle to the distal side of the surface;
movably disengaging the stylet from the needle and advancing the stylet through the needle to extend a first length of a coilable filament coupled at a distal end of the stylet through the tip of the needle, whereupon exiting the tip of the needle the first length of coilable filament forms a first coil on the distal side of the surface;
retracting the needle and rotating the stylet to cause the tip of the needle to withdraw to a proximal side of the surface and to deploy the first segment of the coil along the distal side of the surface; and
movably disengaging the stylet from the needle and advancing the stylet to extend a second length of the coilable filament through the tip of the needle, whereupon exiting the tip of the needle the second length of the coilable filament coils to form a second coil on the proximal side of the surface.

19. The method of claim 16, further comprising releasing the coilable filament to leave the first coil and the second coil in place on the distal and proximal sides of the surface.

20. The method of claim 18, further comprising:
extending the needle through a sheath that conveys the needle to the proximal side of the surface; and
withdrawing the sheath from the distal side of the surface before retracting the needle and rotating the stylet to cause the tip of the needle to withdraw to a proximal side of the surface and to deploy the first segment of the coil along the distal side of the surface.
